(19) United States
(12) Patent Application Publication
(10) Pub. No.: US 2025/0299798 A1
(43) Pub. Date: Sep. 25, 2025

(54) EXTERNAL BEAM RADIATION THERAPY DOSE PREDICTION SYSTEM THROUGH LATENT DIFFUSION MODEL

(71) Applicant: Ever Fortune.AI CO., Ltd., Taichung City (TW)

(72) Inventor: TI-HAO WANG, Taichung City (TW)

(73) Assignee: Ever Fortune.AI CO., Ltd., Taichung City (TW)

(21) Appl. No.: 19/030,488

(22) Filed: Jan. 17, 2025

Related U.S. Application Data
(60) Provisional application No. 63/567,536, filed on Mar. 20, 2024.

(30) Foreign Application Priority Data
Mar. 26, 2024 (TW) 113203046

(51) Int. Cl. G16H 20/40

(52) U.S. Cl. CPC G16H 20/40

(57) ABSTRACT

An external beam radiation therapy dose prediction system through a latent diffusion model is adapted to predict an external beam radiation therapy plan according to a clinical data of a patient and includes a storage unit adapted to store a training data and the clinical data and a processor signally connected to the storage unit. The training data includes a plurality of medical images, a plurality of dose distribution data, and a plurality of training prompts. The processor is adapted to input the training data to a latent diffusion training model to execute a latent diffusion model training and generate an external beam radiation therapy plan model and input the clinical data and at least one prompt to the external beam radiation therapy plan model to generate an external beam radiation therapy plan.

### BACKGROUND OF THE INVENTION

### Technical Field

The present invention relates generally to a radiation therapy, and more particularly to an external beam radiation therapy dose prediction system through a latent diffusion model.

### Description of Related Art

To ensure that a radiation therapy dose could achieve an expected position and an ideal dose and could minimize a damage to healthy organs and healthy tissues, a conventional radiation therapy plan depends on the experience of medical physicists and the experience of dosimetrists for the manual adjustment, thereby consuming time and causing an inaccurate therapy area and an ineffective or inconsistent therapy effect because of the difference of the personal experience. Therefore, the conventional radiation therapy plan still has room for improvement.

Currently, although it had been researched that the radiation therapy plan is improved through a machine learning, anatomical data of patients could not be fully used and an ideal optimization effect is less likely to be achieved in practical application. In addition, although an automatic generation system of a radiation therapy dose distribution by a conditional generative adversarial network had been proposed, the system could not be adapted to different clinical conditions to design the radiation therapy plan, e.g., different therapy instruments (LINAC, Proton, Carbon, Brachytherapy, etc.), different radiation sources (photon, electron, proton, etc.), different therapy techniques (3D-CRT, IMRT, VMAT, etc.).

### BRIEF SUMMARY OF THE INVENTION

In view of the above, the primary objective of the present invention is to provide an external beam radiation therapy dose prediction system through a latent diffusion model, which could predict an external beam radiation therapy plan corresponding to different clinical conditions.

The present invention provides an external beam radiation therapy dose prediction system through a latent diffusion model adapted to predict an external beam radiation therapy plan according to a clinical data of a patient and including a storage unit and a processor, wherein the storage unit is adapted to store a training data and the clinical data. The training data includes a plurality of medical images, a plurality of dose distribution data, and a plurality of training prompts. The processor is signally connected to the storage unit and is adapted to execute the following steps: inputting the training data to a latent diffusion training model to execute a latent diffusion model training and generate an external beam radiation therapy plan model and inputting the clinical data and at least one prompt to the external beam radiation therapy plan model to generate the external beam radiation therapy plan.

With the aforementioned design, based on the clinical data of the patient and the at least one prompt commanded by a clinical staff, the external beam radiation therapy plan could be quickly generated and could be utilized to generate a subsequent therapy plan, thereby raising an efficiency and raising a plan quality.

### BRIEF DESCRIPTION OF THE SEVERAL VIEWS OF THE DRAWINGS

The present invention will be best understood by referring to the following detailed description of some illustrative embodiments in conjunction with the accompanying drawings, in which

FIG. 1 is a block diagram of the external beam radiation therapy dose prediction system through the latent diffusion model according to an embodiment of the present invention; and

FIG. 2 is a flow chart of the external beam radiation therapy dose prediction system through the latent diffusion model according to the embodiment of the present invention.

### DETAILED DESCRIPTION OF THE INVENTION

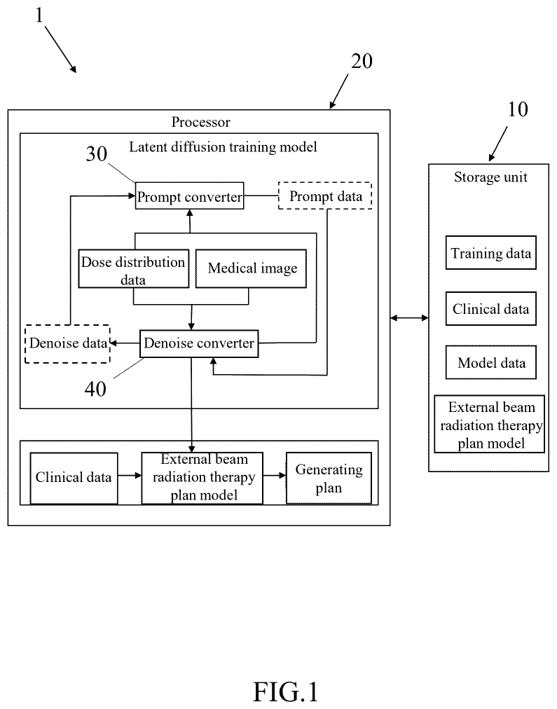

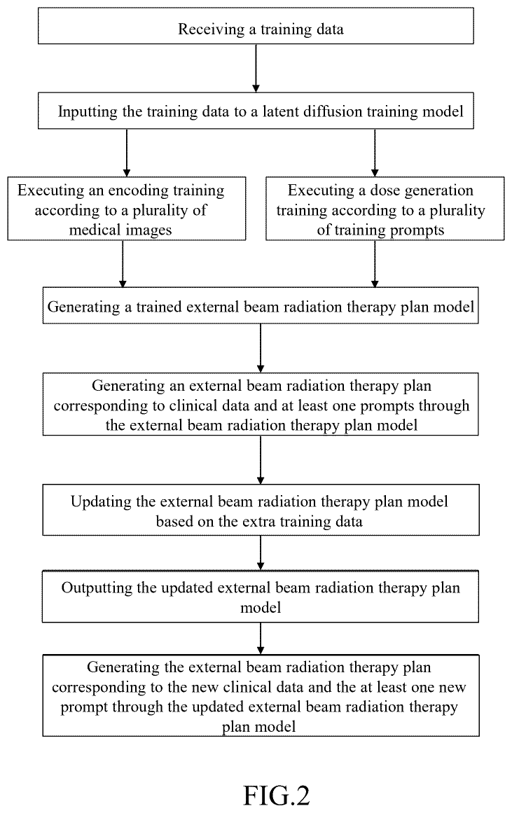

An external beam radiation therapy dose prediction system through a latent diffusion model (LDM) 1 according to an embodiment of the present invention is illustrated in FIG. 1 and FIG. 2 and is adapted to predict an external beam radiation therapy plan according to a clinical data of a patient. The external beam radiation therapy dose prediction system through the latent diffusion model 1 includes a storage unit 10 and a processor 20, wherein the storage unit 10 is adapted to store a training data and the clinical data and could further store a machine learning algorithm and subsets of the machine learning algorithm. The training data includes a plurality of medical images, a plurality of dose distribution data, and a plurality of training prompts, but not limited thereto. The storage unit 10 could be, but not limited to, a memory or another data store component. The medical images include anatomical image data of the patient which could be a magnetic resonance imaging (MRI), a computed tomography (CT) scan, a positron emission tomography (PET), a single photon emission computed tomography (SPECT), or a combination thereof, but not limited thereto. The medical images include a plurality of clinical target contours and a plurality of adjacent organ-at-risk contours. A typical case includes brain cancer, nasopharyngeal cancer, lung cancer, esophageal cancer, breast cancer, cervical cancer, rectal cancer, or prostate cancer, but not limited thereto. The dose distribution data includes 2D radiation therapy dose distribution data or 3D radiation therapy dose distribution data based on the different cases, but not limited thereto. The training prompts include a clinical target and a clinical condition, wherein the clinical target includes an organ or a tissue, but not limited thereto. The clinical condition includes a selected therapy instrument, a radiation source, a therapy technique, a radiation dose, a dose constraint of the clinical target, or a combination thereof.

The processor 20 is signally connected to the storage unit 10 and is adapted to execute a model training of the system 1 and a model generation of the system 1. The processor 20 could be, but not limited to, a central processing unit (CPU), a microprocessor unit (MPU), an application processor (AP), a digital signal processor (DSP), a graphic processing unit (GPU), or a tensor processing unit (TPU). The processor 20 is adapted to execute steps shown in FIG. 2. The steps includes reading the training data stored in the storage unit 10; inputting the training data to a latent diffusion training model; executing an encoding training according to the medical images of the training data and executing a dose generation training according to the training prompts of the training data; generating a trained external beam radiation therapy plan model. After the encoding training and the dose generation training are completed and a new patient data is obtained, the clinical data corresponding to the patient and at least one prompt could be inputted to the external beam radiation therapy plan model to generate the external beam radiation therapy plan corresponding to the patient for the reference of clinicians. For example, the external beam radiation therapy plan includes a radiation therapy dose and a distribution chart of the radiation therapy dose which could be the reference for designing a therapy plan, thereby raising an efficiency of a radiation therapy plan and an accuracy of the radiation therapy plan.

The clinical data includes a plurality of medical images of the patient and a medical record of the patient, but not limited thereto. The at least one prompt includes the clinical target, a disease, a therapeutic factor, a category of the therapy instrument, a category of the radiation source, a therapy, the therapy technique, the radiation dose, and a radiation dose constraint, or a combination thereof. A file format of the at least one prompt could be text, audio, image, video, but not limited thereto. Model data generated during a process of the encoding training and the dose generation training could be stored in the storage unit 10.

Preferably, before a latent diffusion model training is executed, the processor 20 could execute a preprocessing involving the medical images of the clinical data, so that the medical images of the clinical data could satisfy an input format of the external beam radiation therapy plan model. In addition, because noises of the medical images of the clinical data could be reduced, the latent diffusion training model could accurately extract features and a computational burden of the latent diffusion training model could be reduced. For example, the preprocessing includes standardization, cutting, alignment, and data augmentation, but not limited thereto.

The latent diffusion training model includes a prompt converter 30 and a denoise converter 40, wherein the prompt converter 30 is adapted to receive the dose distribution data and a denoise data of the denoise converter 40 to output a prompt data. The denoise converter 40 is adapted to receive the medical images of the clinical data, the dose distribution data, and the prompt data to output the denoise data. The prompt converter 30 could be, but not limited to, a decoder which could decode the at least one prompt which is received to output the prompt data. The denoise converter 40 could be, but not limited to, an encoder with a denoise model adapted to generate the denoise data based on the prompt data of the prompt converter 30 (e.g., decoding result), the dose distribution data, and the medical images of the clinical data.

In addition, the external beam radiation therapy plan model could be updated based on the extra training data to output the updated external beam radiation therapy plan model and the updated external beam radiation therapy plan model could be stored in the storage unit 10. Therefore, through the updated external beam radiation therapy plan model, the external beam radiation therapy plan could be generated based on the new clinical data and the at least one new prompt which are inputted.

Therefore, compared with a generating process of a conventional radiation therapy plan depends on the experience of medical professionals and manual adjustment of the medical professionals, the external beam radiation therapy dose prediction system through the latent diffusion model 1 of the present invention is provided with that the multidimensional anatomical image data of the patient and the at least one prompt commanded by the clinicians are inputted to the external beam radiation therapy plan model through an automatic calculation process. In other words, the medical images of the patient, the clinical target, the clinical target contours and the adjacent organ-at-risk contours, and the at least one prompt commanded by the clinicians are inputted to the external beam radiation therapy plan model. Through the trained external beam radiation therapy plan model, the ideal distribution chart of the radiation therapy dose corresponding to the particular anatomical image data of the patient is generated, thereby providing a reference of a subsequent therapy plan to the clinician to reduce a time of designing the therapy plan and raising the efficiency of the radiation therapy plan.

It must be pointed out that the embodiments described above are only some preferred embodiments of the present invention. All equivalent structures which employ the concepts disclosed in this specification and the appended claims should fall within the scope of the present invention.

What is claimed is:

1. An external beam radiation therapy dose prediction system through a latent diffusion model adapted to predict an external beam radiation therapy plan according to a clinical data of a patient and comprising:
a storage unit adapted to store a training data and the clinical data, wherein the training data comprises a plurality of medical images, a plurality of dose distribution data, and a plurality of training prompts;
a processor signally connected to the storage unit and adapted to execute the following steps:
imputing the training data to a latent diffusion training model to execute a latent diffusion model training and generate an external beam radiation therapy plan model;
inputting the clinical data and at least one prompt to the external beam radiation therapy plan model to generate the external beam radiation therapy plan.

2. The external beam radiation therapy dose prediction system through the latent diffusion model as claimed in claim 1, wherein the latent diffusion model training comprises executing an encoding training according to the plurality of medical images and executing a dose generation training according to the plurality of training prompts.

3. The external beam radiation therapy dose prediction system through the latent diffusion model as claimed in claim 1, wherein the processor executes a preprocessing involving a plurality of medical images of the clinical data, so that the medical images of the clinical data could satisfy an input format of the external beam radiation therapy plan model.

4. The external beam radiation therapy dose prediction system through the latent diffusion model as claimed in claim 1, wherein the plurality of training prompts comprise a clinical target and a clinical condition.

5. The external beam radiation therapy dose prediction system through the latent diffusion model as claimed in claim 4, wherein the clinical condition comprises a therapy instrument, a radiation source, a therapy technique, a radiation dose, a dose constraint of the clinical target, or a combination thereof.

6. The external beam radiation therapy dose prediction system through the latent diffusion model as claimed in claim 1, wherein the plurality of medical images comprise a magnetic resonance imaging, a computed tomography scan, a positron emission tomography, a single photon emission computed tomography, or a combination thereof.

7. The external beam radiation therapy dose prediction system through the latent diffusion model as claimed in claim 1, wherein the plurality of medical images comprise a plurality of clinical target contours and a plurality of adjacent organ-at-risk contours.

8. The external beam radiation therapy dose prediction system through the latent diffusion model as claimed in claim 1, wherein the latent diffusion training model comprises a prompt converter and a denoise converter; the prompt converter is adapted to receive the plurality of dose distribution data, a denoise data of the denoise converter to output a prompt data; the denoise converter is adapted to receive the plurality of medical images, the plurality of dose distribution data, and the prompt data to output the denoise data.
